(19) United States
(12) Patent Application Publication
(10) Pub. No.: US 2013/0324914 A1
(43) Pub. Date: Dec. 5, 2013

(54) SWALLOWABLE CAPSULE FOR INSUFFLATION OF GASTROINTESTINAL TRACT

(71) Applicants: Pietro Valdastri, Nashville, TN (US); Gastone Ciuti, Pisa (IT); Arianna Menciassi, Pisa (IT); Paolo Dario, Livorno (IT); Robert J. Webster, III, Nashville, TN (US); Byron F. Smith, Memphis, TN (US); Jenna L. Gurlewicz, Nashville, TN (US)

(72) Inventors: Pietro Valdastri, Nashville, TN (US); Gastone Ciuti, Pisa (IT); Arianna Menciassi, Pisa (IT); Paolo Dario, Livorno (IT); Robert J. Webster, III, Nashville, TN (US); Byron F. Smith, Memphis, TN (US); Jenna L. Gurlewicz, Nashville, TN (US)

(73) Assignee: VANDERBILT UNIVERSITY, Nashville, TN (US)

(21) Appl. No.: 13/818,969

(22) PCT Filed: Aug. 26, 2011
(86) PCT No.: PCT/EP2011/064764

§ 371 Date: Aug. 1, 2013

(30) Foreign Application Priority Data
Aug. 30, 2010 (IT) FI2010A000182

(51) Int. Cl. A61M 13/00; A61B 1/00

(52) U.S. Cl. CPC A61M 13/003; A61B 1/00158
USPC 604/26

(57) ABSTRACT

A system for insufflating a body cavity comprising a swallowable capsule comprising a chamber (11) containing a fluid capable to generate biocompatible gas or vapour by phase transition or chemical reaction, the chamber (11) being formed with at least one orifice (7) for placing it in communication with the outside. The orifice (7) is blocked by a shutter (4, 6; 26) made of magnetic material and there are provided elastic means (8) able to generate an elastic force sufficient to keep said shutter sealingly engaged in the orifice. The system further comprises magnetic actuating means (12; 34) external to the body cavity for generating a magnetic force having strength sufficient to overcome the elastic force of the elastic means disengaging the shutter from the orifice.

### FIELD OF THE INVENTION

The present invention relates to the field of endoscopy, and in particular the endoscopy of the gastrointestinal tract; more precisely it refers to a system for insufflation of the gastrointestinal tract. More in particular the invention aims at providing a swallowable capsule for insufflation of the gastrointestinal tract.

### STATE OF THE ART

The endoscopic procedures for exploring the interior cavities and articulations of the human body have been used almost thirty years and they are conventionally of the fibre optic endoscopy type provided with powerful lens systems and with at least one light source for illuminating the areas of interest. In the case of colon endoscopy the movement of the endoscope is facilitated by the introduction of air, or insufflation, from an external source to distend the tissues, otherwise collapsed, of the colon and allow an accurate visualisation thereof. These procedures have the drawback of being painful for the patient and they have the limit of not allowing the visualisation of the small intestine.

This limit was overcome through the introduction of swallowable and passively mobile endoscopic capsules into the gastrointestinal tract due to the peristaltic movements thereof. The endoscopic capsules allow obtaining images of areas of interest of the gastrointestinal tract in a minimally invasive manner and without causing pain to the patient. The images thus obtained are used by the physician to detect lesions, polyps, internal bleeding areas or for an early diagnosis of cancer of the gastrointestinal tract. The market leader in the endoscopic capsules industry is Given Imaging through the PillCam® System products. Other important producers of endoscopic capsules are Olympus (Endocapsule), IntroMedic Co. (MiroCam®) and Chongqing Jinshan Science & Technology Group Co. Ltd (JS-MEII OMOM).

The success of the commercial capsules with passive locomotion lead to the development of various researches aimed at improving the diagnostic capacity thereof providing them with robotic functionalities, in particular to allow active locomotion thereof and to allow direct intervention thereof to treat the detected lesions. A very important aspect for the operation of a robotised capsule lies in the capacity thereof to distend the tissues of the traversed gastrointestinal tract with the aim of preventing them from hindering the locomotion of the capsule and allow a suitable visual exploration thereof. The need to distend the tissues also arises in the case of capsules with passive locomotion when they are used for exploring gastrointestinal system tracts, such as the colon, where reliability thereof is quite low due to the tendency of the tissues to collapse.

Two solutions were essentially proposed for distending the tissues of the gastrointestinal tract: the first envisages the use of a mechanical device provided with extendable legs actuated by direct current motors, the second provides for the use of a fluid chemical system through which a section of the gastrointestinal tract is insufflated. If, on one hand, the first system allows both the locomotion of the capsule and the distending of the surrounding tissue, on the other hand it however requires a complex, expensive and high energy consumption electromechanical device. Though the fluid chemical systems have a much lower energy demand for the operation thereof, the insufflation capacity of these systems are limited by volumetric constraints of the capsule and by the volume occupied within the capsule by the various components required to generate and control the insufflation.

The first solution is represented by a capsule with twelve legs described in WO2008/122997. The capsule is provided with two groups of six radially extensible legs which allow a uniform distension of the collapsed tissue of the colon thus facilitating the visualisation of the interior surface and simultaneously allowing the autonomous locomotion of the capsule. Even if the capsule according to the aforementioned patent revealed to be capable of moving over the entire length of the large intestine overcoming the narrow curves, such as the splenic flexure of the colon, it shows various technical drawbacks regarding the supply system.

The second solution is suggested by Toennies J. L. et al. in “A wireless insufflation system for capsular endoscopes”, ASME J. Medical Devices, 2009, 3(2), 27514. The capsule includes an insufflation system whose operation principle is based on the generation of a relatively high volume of gas following the catalytically induced dissociation of a fluid carried inside the capsule. The prototype of the capsule for insufflation comprises the following components: a tank for the fluid, a one-way solenoid valve, a wireless communication electronic unit, a silver mesh catalyst and a battery. Hydrogen peroxide (70% concentration) was selected as the fluid due to its high expansion volumetric ratio following catalytic dissociation, hence allowing integrating all the components in a capsule whose dimensions can be compared to that of an endoscopic capsule available in the market, i.e. about 11 mm of diameter and 24-31 mm (averagely 26 mm) of length.

The possibility of developing a valid system of insufflation capable to distend the collapsed tissue of the colon has a double clinical value for an endoscopic capsule: (a) potential improvement of the visualisation of the lumen through the on board camera, and (b) an easier passage of capsules using active locomotion strategies. The fluidic system for generating a relatively high gas volume suggested by Toennies et al. has a complex activation system which reduces the volume available for the fluid and thus the capacity of the capsule to generate—within the colon—a pressure sufficient to cause a satisfactory distension of the tissue.

### SUMMARY OF THE INVENTION

Subject of the present invention is to provide a system for the insufflation of a body cavity, such as the gastrointestinal tract, capable of leading to a suitable production of gas coming from a fluid contained in a swallowable capsule.

Another subject of the present invention is to provide an insufflation system of the aforementioned type in which the capsule has an internal volume available for containing the fluid, greater than that allowed according to the known art.

A further subject of the present invention is to provide a system of the aforementioned type in which it is possible to position and orient the capsule within the body cavity.

These subjects are attained by means of the system for insufflation of a body cavity according to the present invention whose essential characteristics are indicated in claim 1. Further important characteristics are contained in dependent claims.

### BRIEF DESCRIPTION OF THE DRAWINGS

Further characteristics and advantages of the insufflation system according to the present invention will be clear from the following description of an embodiment thereof provided by way of non-limiting example with reference to the attached drawings wherein:

FIG. 1 is a perspective view of the insufflating capsule part of the insufflation system according to the present invention;

FIG. 2 is a longitudinal view of the capsule of FIG. 1;

FIG. 3 is a longitudinal view of the capsule of FIG. 1 from the side of the outlet orifice;

FIG. 4 is a perspective cross-sectional view according to arrows IV-IV of FIG. 3 of the insufflating capsule according to the invention;

FIGS. 5 and 6 show—in longitudinal section—the effect of approaching a permanent external magnet to the insufflating capsule of the preceding figures;

FIGS. 7 and 8 show a longitudinal section of a different embodiment of the insufflating capsule part of the insufflation system according to the invention, and of the related external magnetic actuator device in the position of orienting the capsule and, respectively, of activating the insufflation;

FIG. 9 is a detail section of a further variant of the insufflating capsule of the insufflation system according to the present invention.

### DETAILED DESCRIPTION OF THE INVENTION

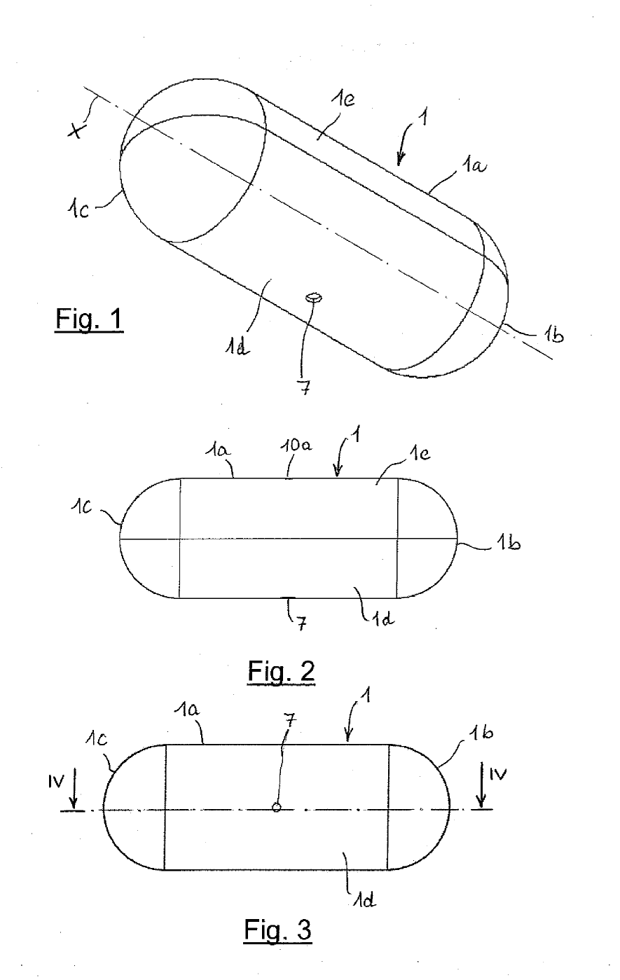

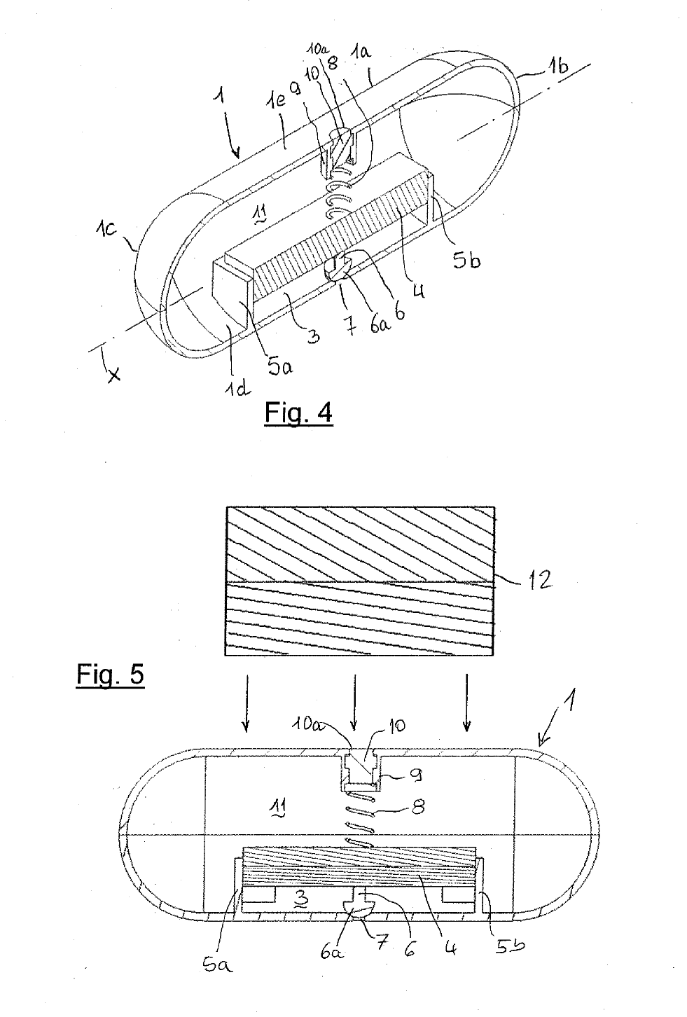

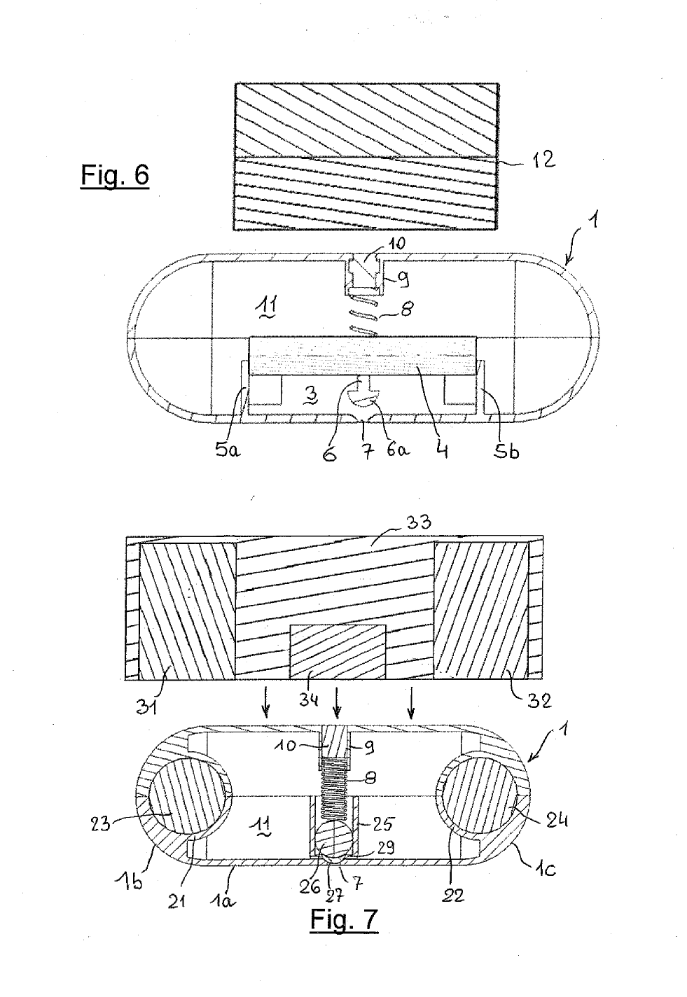

With reference to FIGS. 1 to 6, a body of the swallowable capsule part of the insufflation system according to the invention, of hollow cylindrical shape and closed at the ends, is indicated with 1. In particular, the capsule body 1 is formed by a tubular portion 1a closed by two cap ends 1b and 1c and it is preferably made of two equal halves 1d and 1e, welded to each other along the respective edges. X is used to indicate the longitudinal axis along which the capsule body 1 extends.

The capsule body 1 is made of a biocompatible material resistant to gastric acids, for instance polyolefin materials, such as high density polyethylene (HDPE), or fluoropolymer materials, such as polytetrafluoroethylene (PTFE). The thickness of the capsule body has to resist to an internal pressure in the order of 50 kPa. The dimensions of the capsule are such as to allow an easy swallowing thereof, for example such as for a vitamin pill or commercial endoscopic capsules.

In one of the two halves 1d, at a central and symmetric position with respect to the lateral edges thereof, there is provided a seat 3 in which a permanent magnet 4 is arranged, substantially parallelepiped shaped with axial magnetization direction. Permanent magnets having different shapes, for example cylindrical-shaped, may be used alternatively. The seat 3 is in particular defined by two opposite walls 5a and 5b, substantially C-shaped, in which the magnet 4 is positioned in a radially slidable manner. The two walls 5a and 5b extend radially from the internal face of the half 1d.

From the side of magnet 4 opposite to the longitudinal axis X there centrally and radially extends a stem 6 terminating with an enlarged head 6a which is sealingly engaged in an orifice 7 which traverses the wall of the half 1d. On the opposite side of the magnet 4, aligned with the stem 6, a helical spring 8 forces, that abuts with the opposite end thereof against an abutment element 10 arranged at the bottom of a tubular seat 9 in which there is engaged an end portion of the spring 8. The seat 9 radially extends from the internal face of the other half le of the capsule body 1, with the function of guaranteeing a suitable constraint for the end of the spring 8 engaged therein and the abutment element 10 is sealingly engaged in an orifice 10a obtained on the capsule body 1a on the diametrically opposite side with respect to the orifice 7.

The capsule body 1 delimits on the inside a chamber 11 filled with a fluid adapted to generate a relatively large volume of gas following a thermally induced phase transition. Perfluoropentane, a biocompatible liquid substance at room temperature and which evaporates at body temperature, is used in the present embodiment of the invention. This substance has a boiling temperature of 29° C. at atmospheric pressure and a liquid/vapour volumetric conversion ratio at 37° C. of about 1/100 at atmospheric pressure.

The spring 8 exerts an elastic force on the magnet 4 sufficient to ensure a sealing engagement of the enlarged head 6a of the stem 6 in the orifice 7 and the entirety forms a block magnetic valve in which the stem 6 and the relative enlarged head 6a serve as a shutter made of ferromagnetic material and the orifice 7 as a valve seat. Under these conditions the liquid contained within the capsule 1 is prevented from flowing out.

Instead, by applying an external magnetic field through a permanent magnet 12 generating a magnetic force sufficient to overcome the elastic reaction of the spring 8, the magnet 4 slides in its seat 3, and the enlarged head 6a of the stem 6 is disengaged from the orifice 7 placing the chamber 11 of the capsule body 1 in communication with the surrounding environment and thus allowing the exit of the gas generated by the fluid contained therein.

During use, upon swallowing the capsule, the path of the capsule may be followed by means of any known localisation system (for example magnetic, ultrasonic or radiofrequency or by processing images of the gastrointestinal tract), until it reaches the predetermined site inside the gastrointestinal tract. In the meanwhile, also a conventional endoscopic capsule, with passive or active locomotion, has been swallowed by the patient and the activation of the insufflation capsule may be controlled when the endoscopic capsule has also reached the gastrointestinal tract. At this point, the activation of an external magnetic field causes the disengagement of the shutter 6a from the orifice 7 and the gas generated by the fluid contained in the chamber 11 can flow through the orifice 7, hence the gas is released into the intestinal cavity with consequent distension of the tissues.

It should be observed that, once the capsule reaches the desired area, an external magnetic field can be used to move the capsule to a precise position and suitably orient it in a direction aligning the external magnetic field when needed. During this process, the capsule automatically aligns with the external magnetic field and thus the internal single magnet 4 aligns towards the external magnet 12, depending on the choice of a suitable and analogous magnetization direction of the aforementioned magnets 4 and 12. By approaching the external magnet 12 to the patient body, the magnetic force acting on the internal magnet 4 progressively increases in strength until the elastic reaction of the spring 8 is overcome, and the spring 8 is compressed thus opening the orifice 7.

The capsule can be localised by means of an array of magnetostrictive sensors or Hall effect sensors arranged outside, suitably referred with respect to each other, and allowing locating the capsule in a system of absolute coordinates referring to the external sensors. Examples of solutions of this type are already available on the market. See for example the systems described in H. Richert et al., “Magnetic sensor techniques for new intelligent endoscopic capsules” (Conference paper) 10th Symposium Magnetoresistive Sensors and Magnetic Systems, 31 Mar.-1 Apr. 2009 at Wetzlar, available in the Internet page http://www.vector-project.com/press/artikel/VECTOR%

0article_Richert_MagneticSensorTechniques.pdf

It should be observed that the abutment element 10 can be made of elastomeric material with high degree of hardness, to guarantee the possibility of a subsequent filling of the chamber 11 with a fluid through a simple injection procedure using a needle of a filling device once the capsule has been constructed entirely. This solution may also allow a potential reutilisation of the capsule.

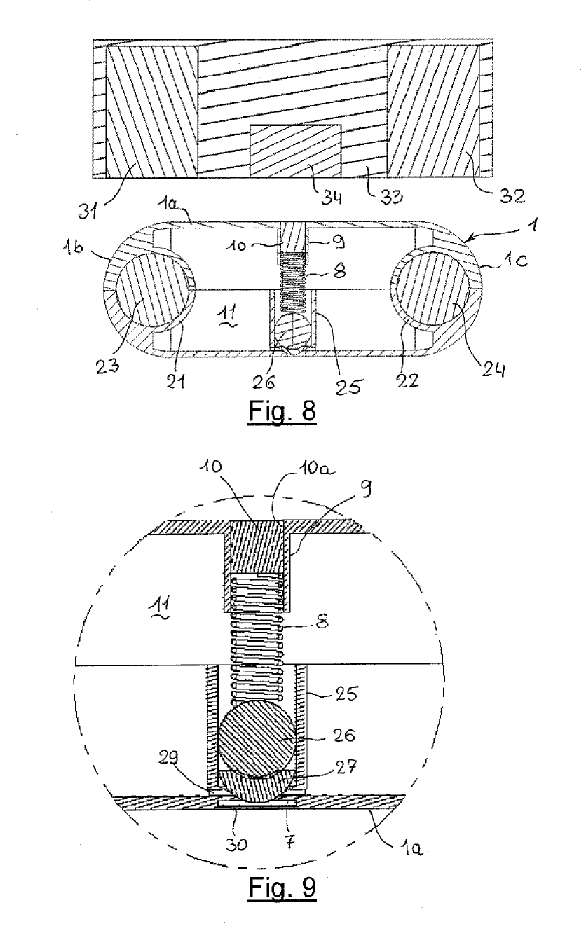

An insufflating capsule which offers better possibilities of orientation, though having a smaller available internal volume with respect to the capsule according to the previously described embodiment, is illustrated in FIGS. 7 and 8. In the insufflating capsule illustrated in these figures (in which identical components are indicated with the same reference numbers of FIGS. 1-6) there are provided separated magnetic means for performing the functions of orienting the capsule and, respectively, actuating the shutter. In particular, at the two ends of the capsule body 1 there are provided two seats 21 and 22 in which there are housed two magnets 23 and 24, for example spherical-shaped.

Around the orifice 7 there is formed a tubular seat 25 radially extended in which there is arranged a third permanent magnet 26 against which there abuts the spring 8 to keep it abutting against the orifice 7 through a gasket 27, fixed to the magnet, which is sealingly engaged therein. Between the tubular seat 25 and the wall of the capsule body 1 there are provided openings 28 in order to allow the passage of the fluid when the magnet 26 moves away from the orifice 7 due to a magnetic field having strength sufficient to overcome the elastic force generated by the spring 8 on the magnet 26.

In this case, an orienting system made up of two external permanent magnets 31 and 32 with direction of magnetisation and relative distance analogous to that of the two internal magnets 23 and 24 arranged at the ends of the capsule body 1, is used for orienting the capsule. The two magnets 31 and 32 are housed in a handpiece 33 together with a permanent magnet 34, arranged at an intermediate position between the two magnets 31 and 32 in the handpiece 33, which has the function of activating the magnetic valve. During the process of orientation, the magnetic fields inside the capsule automatically align with the magnetic fields of the orienting system, and guarantee the correct orientation of the device for the subsequent disengagement of the shutter, opening the orifice and subsequent exit of the gas generated by the fluid contained therein. The arrangement of the magnetic poles at the ends of the capsule and the arrangement of the poles of the external magnets guarantees the actual orientation of the capsule, as if virtual anchoring constraints be created. In this case, the capsule is oriented both regarding the ROLL angle and regarding the YAW angle.

Also in this case, after having oriented the capsule in the desired manner, approaching the handpiece 33 to the body cavity where the capsule is located at that moment generates, through the central magnet 34, a magnetic attraction force on the magnet 26 sufficient to overcome the resistance of the spring 8 causing the disengagement of the magnet 26 from the orifice 7.

Other fluids can be used for generating the gas volume required to distend the tissues of the body cavity to be explored. Without prejudice to their biocompatibility, these fluids must have a phase transition from liquid to vapour at a temperature not lower than the room temperature and not exceeding the mean body temperature at the operating pressure of the capsule. Alternatively, the formation of a gaseous phase may be catalytically induced, possibly through a disassociation reaction. In this case, the fluid is passed on a bed of a suitable catalyst when exiting from the capsule. For example, using hydrogen peroxide at 70% in liquid phase and a silver catalytic bed, the hydrogen peroxide is dissociated in oxygen and water vapour with considerable increase of the volume of the gaseous phase.

A possible embodiment of the insufflating capsule using hydrogen peroxide is shown in detail in FIG. 9. The capsule is structurally identical to the one illustrated in FIGS. 7 and 8, but at the orifice 7 there is provided a silver net 30, between whose meshes the hydrogen peroxide passes being dissociated upon contact.

In the present description, when reference is made to relatively high volumes of gas and/or vapours produced by the fluid contained in the capsule, either generated following a fluid transition phase, for example induced thermally or in any other manner, or generated following a chemical reaction, for example induced catalytically, it should be understood that said fluid is characterized by a vapour/liquid or gas/liquid or vapour-gas/liquid volumetric ratio of at least 50 and preferably of at least 100, and more generally such as to produce a distension of the tissues of the gastrointestinal tract under inspection sufficient to allow the locomotion of the capsule and the accurate visualisation of the tissues.

The system for insufflation of the gastrointestinal tract according to the present invention may be subjected to variants and/or modifications without departing from the scope of protection of the invention as defined in the attached claims.

1. A system for insufflation of a body cavity comprising a swallowable capsule comprising a chamber containing a fluid capable of generating biocompatible gas and/or vapour by phase transition or chemical reaction, said chamber being formed with at least one orifice for communicating with the outside of the capsule, said at least one orifice being blocked by a shutter of ferromagnetic material and being provided elastic means for generating an elastic force sufficient to keep said shutter sealingly engaged in said orifice, said system further comprising magnetic actuating means external to said body cavity for generating a magnetic force having a strength sufficient to overcome the elastic force of said elastic means, thereby disengaging said shutter from said orifice.

2. The system according to claim 1, wherein said capsule extends along a longitudinal axis (X) and wherein in said capsule, at said orifice, a seat is formed perpendicularly extending to said axis and in which said shutter in ferromagnetic material is slidingly arranged.

3. The system according to claim 1, wherein said shutter in ferromagnetic material comprises a permanent magnet from which a shutter extends engaged with said orifice, said elastic means acting on said magnet at the opposite part of said shutter.

4. The system according to claim 3, wherein said magnet is responsive to the external magnetic field generated by said external magnetic actuating means for orienting the capsule within said body cavity.

5. The system according to claim 1, wherein said shutter in ferromagnetic material comprises an internal permanent magnet elastically engaged in said orifice.

6. The system according to claim 5, wherein a pair of inner permanent magnets is arranged at both ends of said capsule body and said external magnetic actuating means comprise two external permanent magnets having magnetization direction and relative distance substantially equal to that of the inner magnets for orienting the capsule within said body cavity, and an external permanent magnet in an intermediate position relative to said two external magnets, for generating a magnetic force on said internal permanent magnet sufficient to overcome the resistance of the elastic means by approaching said external magnetic actuating means to said capsule.

7. The system according to claim 1, wherein the fluid contained in said chamber is capable of producing gas by thermally induced phase transition.

8. The system according to claim 7, wherein said fluid is perfluoropentane.

9. The system according to claim 1, wherein the fluid contained in said chamber is capable of producing gas by chemical reaction.

10. The system according to claim 9, wherein said chemical reaction is a catalytically induced dissociation reaction.

11. The system according to claim 10, wherein in said chamber a catalytic bed is provided through which said fluid passes before being released out from said orifice.

12. The system according to claim 11, wherein said catalytic bed is arranged at said orifice.

13. The system according to claim 9, wherein said fluid is hydrogen peroxide and said catalytic bed is a silver mesh.

14. The system according to claim 6, wherein a seat for sliding said magnetic shutter is provided, said seat of said magnetic shutter comprising passages for placing it into communication to said chamber.

15. The system according to claim 1, wherein said elastic means, at the opposite side of said magnetic shutter abuts on an abutment member made of hard elastomeric material pierceable from the outside with a needle of a filling device.
